(19) United States
(12) Patent Application Publication
(10) Pub. No.: US 2008/0233179 A1
(43) Pub. Date: Sep. 25, 2008

(54) TRANSDERMAL COMPOSITION HAVING ENHANCED COLOR STABILITY

(76) Inventors: Arnaud GRENIER, Steinbrunn le Haut (FR); Dario Norberto R. CARRARA, Oberwil (CH)

Correspondence Address: WINSTON & STRAWN LLP;PATENT DEPARTMENT, 1700 K STREET, N.W., WASHINGTON, DC, 20006, US

(21) Appl. No.: 11/770,269

(22) Filed: Jun. 28, 2007

Related U.S. Application Data
(60) Provisional application No. 60/818,088, filed on Jun. 29, 2006.

(51) Int. Cl. A61K 31/404; A61K 9/70

(52) U.S. Cl. 424/449; 514/418

(57) ABSTRACT

A pharmaceutical composition for transdermal or transmucosal delivery of a pharmaceutical agent effective to treat a neurological disorder such as Parkinson's disease is disclosed. The composition contains an antioxidant, which provides color and chemical stability of the pharmaceutical agent. The antioxidant may also provide enhanced skin permeability of the agent.

This application claims the benefit of provisional application 60/818,088 filed Jun. 29, 2006, the entire content of which is expressly incorporated herein by reference thereto.

### FIELD OF THE INVENTION

The invention relates generally to transdermal drug delivery, and more particularly to transdermal compositions and methods of administering an active agent. The invention additionally relates to a non-occlusive transdermal semi-solid composition containing an active agent which is chemically stable and which provides enhanced permeation through the skin or the mucosa.

### BACKGROUND OF THE INVENTION

Parkinson's disease (PD) is a hypokinetic disorder comprised of four features: (i) bradykinesia (slowness and poverty of movement); (ii) muscular rigidity (an increase in the resistance of the muscles to passive movement); (iii) resting tremor; and (iv) abnormalities of posture and gait. In Parkinson's disease, the dopaminergic system is deficient due to the degeneration of dopaminergic neurones in the nigrostriatal pathway, which allows the cholinergic system to hold unopposed sway, resulting in abnormal control of muscular activity. Thus, the two main approaches to treating Parkinson's disease have been replenishment of the stores of dopamine and reduction of excessive cholinergic action by acetylcholine antagonists. While it is difficult to estimate the number of people affected by this disease, because the symptoms of the disease are often mistaken for the normal results of aging or are attributed to other diseases, Parkinson's disease occurs in people all over the world, in all ages.

Presently, the most effective anti-Parkinson drug available is levodopa. When levodopa is taken alone, the body breaks down about 95% of the drug into dopamine before it reaches the brain, producing a lot of side effects. Combining levodopa with another drug such as carbidopa (e.g., SINEMET® of Merck) or benserazide enables more levodopa to enter the brain before it converts into dopamine. As many as half the people who take this drug for two to five years begin to notice fluctuations in the drug's effectiveness, known as an on-off effect. Others develop dyskinesia—involuntary movements such as jerking or twitching. As Parkinson's disease progresses, the effectiveness of the combination also decreases and patients require higher and more frequent doses to control their symptoms.

As an alternative to levodopa, dopamine agonists have played an important role in treating Parkinson's disease. Dopamine agonists, such as pergolide, lisuride and pramipexole, mimic the action of dopamine by activating nerve cells in the striatum. Dopamine agonists are increasingly used alone in the early stages of Parkinson's disease in order to lower a patient's risk of developing the dyskinesia associated with levodopa therapy. Later in the course of the disease they are more likely to be combined with carbidopa or levodopa to alleviate that drug's on-off effects. All available dopamine agonists stimulate D2 receptors, which is believed to be clinically beneficial.

One of the dopamine agonists indicated for the treatment of idiopathic Parkinson's disease is pramipexole, which has become one of the most widely used dopamine agonists because of its proven efficacy. Pramipexole is presently marketed as the hydrochloride salt in an immediate-release tablet for the treatment of Parkinson's Disease (MIRAPEX® of Boehringer Ingelheim). Pramipexole dihydrochloride has the chemical name (S)-2-amino-4,5,6,7-tetrahydro-6-(propylamino)benzothiazole dihydrochloride monohydrate and a structural formula as shown below:

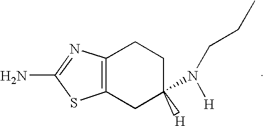

Pramipexole, an indolone compound, is a nonergot dopamine agonist with a high relative in vitro specificity and full intrinsic activity at the D2 subfamily of dopamine receptors, and binds to D3 receptors with higher affinity than to D2 or D4 receptor subtypes. While the precise mechanism of action of pramipexole as a treatment for Parkinson's disease is unknown, it is believed that pramipexole provides treatment by stimulating dopamine receptors in the striatum. This conclusion is supported by electrophysiological studies in animals which demonstrate that pramipexole influences striatal neuronal firing rates via activation of dopamine receptors in the striatum and the substantia nigra, the site of neurons that send projections to the striatum. Pramipexole, its chemical structure, processes for its preparation and therapeutic uses thereof are more fully described in U.S. Pat. Nos. 4,452,808, 4,824,860, and 6,770,761.

Administration of any active pharmaceutical agent, including pramipexole and other anti-Parkinson agents, should preferably be provided by an administration regime—the route of administration and the dose regimen—that is as simple and non-invasive as possible in order to maintain a high level of compliance by the patient. Oral administration is an administration regime that is commonly used because it is relatively simple to follow, but oral administration may cause many side effects and complications, including, among others, complications associated with gastrointestinal irritation and drug metabolism in the liver. For instance, oral administration of pramipexole can cause serious adverse effects such as nausea, dizziness, drowsiness, somnolence, insomnia, constipation, unusual weakness, stomach upset and pain, headache, dry mouth, hallucinations, difficulty moving or walking, difficulty breathing, confusion, restlessness, leg or foot swelling, fainting, twitching, chest pain, unusually fast or slow heartbeat, muscle pain, vision problems, fever, severe muscle stiffness, and sudden irresistible urge to sleep. Even administration of small amounts of pramipexole, which is typically administered at a daily does of about 1.5 to 4.5 mg, with bioavailability of 90%, is associated with considerable side effects. An alternative route of administration is therefore desired.

Recently, administration of active pharmaceutical agents through the skin—the “transdermal drug delivery”—has received increased attention because it provides not only a simple dosage regime but also a relatively slow and controlled release of an active agent into the system, ensuring a safe and effective administration of the active agent. Advantageously, transdermal administration can totally or partially alleviate the side effects associated with oral administration. For example, U.S. Pat. No. 5,112,842 explains that continuous transdermal delivery of pramipexole provides a number of advantages, such as sustained pramipexole blood levels, which is believed to provide a better overall side effect profile than typically associated with oral administration; absence of first-pass effect; substantial avoidance of gastrointestinal and other side effects; and improved patient acceptance.

Transdermal administration of pramipexole by means of a patch, also known as transdermal therapeutic system (TTS), is known. For example, U.S. Patent Application Publication No. US 2004/0253299 discloses a reservoir-TTS containing pramipexole or a pharmaceutically acceptable salt or derivative thereof, and a chelate former or an antioxidant as a stabilizer as applicable, which is stable to decomposition and provides for release of the active ingredient over a period of three or more days. U.S. Patent Application Publication No. US 2006/0078604 discloses a transdermal drug delivery system for topical application of pramipexole, contained in one or more polymeric and/or adhesive carrier layers proximate to a non-drug containing polymeric backing layer, where the delivery rate and profile is controlled by adjusting the moisture vapor transmission rate of the polymeric backing layer. U.S. Pat. No. 6,221,383 discloses a TTS comprising a blend of polymers, which provides a pressure-sensitive adhesive composition for transdermal delivery of drugs.

Transdermal therapeutic systems or patches, however, present many drawbacks, such as skin irritation caused by high drug loading per cm², adhesives used in the patch, and the occlusive nature of the patch. Therefore, a non-patch, non-occlusive composition for transdermal delivery of an anti-Parkinson agent is desired.

Certain non-patch, transdermal compositions containing pramipexole are known. U.S. Pat. No. 6,383,471 discloses a pharmaceutical composition, which comprises (a) a hydrophobic therapeutic agent having at least one ionizable basic functional group and (b) a carrier comprising (i) a pharmaceutically acceptable inorganic or organic acid; (ii) a surfactant selected from the group consisting of non-ionic hydrophilic surfactants having an HLB value greater than or equal to about 10, ionic hydrophilic surfactants, hydrophobic surfactants having an HLB value less than 10, and mixtures thereof; (iii) optionally a triglyceride; and (iv) optionally a solubilizer. U.S. Pat. No. 6,833,478 discloses a method for increasing the solubility of an anti-Parkinson agent in a lipophilic medium, the method comprising admixing the agent with a solubility-enhancing amount of an N,N-dinitramide salt, wherein ionization of the agent results in a biologically active cationic species in association with an anionic counter-ion. Pramipexole is not included as one of the anti-Parkinson agents disclosed in this publication. U.S. Pat. No. 6,929,801 discloses a transdermal drug delivery system comprising a therapeutically effective amount of an anti-Parkinson agent such as pramipexole, at least one dermal penetration enhancer which is a skin-tolerant ester sunscreen, and at least one volatile liquid.

However, because certain anti-Parkinson agents such as pramipexole have poor stability in commonly used pharmaceutical solvents, massive discoloration may rapidly occur in a transdermal delivery composition containing such agents. The chemical and color stability problems of pramipexole are discussed in U.S. Patent Application Publication No. US 2005/0059717, which provides new acid salts of pramipexole with enhanced stability, including more stable coloration, intended for solid dosage forms, especially for oral solid dosage forms. Thus, there is a need for a cosmetically-acceptable transdermal composition of an anti-Parkinson agent which provides enhanced chemical and color stability.

Further, it would be advantageous to provide a transdermal composition of pramipexole which exhibits sustained release of pramipexole such that the composition can be administered once a day. Conventionally, pramipexole is administered several times a day. Hubble et al., Clinical Neuropharmacology 18(4), 338-347 (1995) describes administration of pramipexole three times a day in patients with early Parkinson's disease. Steady-state pharmacokinetic properties of pramipexole, when administered three times a day in the form of pramipexole dihydrochloride tablets as reported in Wright et al., Journal of Clinical Pharmacology 37, 520-525 (1997), concludes that steady-state pharmacokinetic characteristics are linear up to a daily dose of 4.5 mg with such multiple administrations.

U.S. Patent Application Publication No. US 2006/0110454 states that the prior art recognizes reduced side effect profile of once daily dosage form, compared to thrice daily immediate release dosage form. U.S. Patent Application Publication No. US 2005/0226926 also discloses that a three times daily dosing regimen for immediate-release pramipexole dihydrochloride tablets is well tolerated, but that patient compliance would be much improved if a once-daily regimen were possible. Because Parkinson's disease is an affliction that becomes more prevalent with advancing age, a once-daily regimen is noted as especially useful in enhancing compliance among elderly patients. Thus, a once daily administration of an anti-Parkinson agent such as pramipexole would be desirable. Such a composition would simplify the administration regime of the drug by reducing the number of daily application and improve patient compliance, while also reducing adverse events and side effects associated with an immediate release formulation, such as high plasma peaks.

In addition, it would be advantageous to provide a transdermal composition which allows improved permeation of the anti-Parkinson agent while maintaining the stability of the agent in the composition. Although transdermal compositions are generally known, it can be difficult to find a permeation enhancer that is compatible and effective with a particular drug.

Thus, to improve upon prior art formulations, what is needed is a transdermal composition of an anti-Parkinson agent having improved stability and permeation properties, which can be provided in a non-patch or non-occlusive form and which can provide a sustained release of the anti-Parkinson agent.

### SUMMARY OF THE INVENTION

The invention relates to a transdermal, preferably non-occlusive, composition comprising a therapeutically effective amount of a pharmaceutical agent effective to treat a neurological disorder and an antioxidant, wherein the composition exhibits enhanced color stability compared to a composition not containing the antioxidant. The composition provides an effective transdermal therapy for treatment of neurological disorders such as Parkinson's disease, Restless Leg Syndrome, Tourette's Syndrome, Chronic Tic Disorder, Essential Tremor, and Attention Deficit Hyperactivity Disorder. Thus, the invention also relates to a method of treating a neurological disorder by administering such composition.

The pharmaceutical agent can be an indolone dopamine agonist, such as pramipexole or a pharmaceutically acceptable salt of thereof. The pharmaceutical agent can be included in any desired amount, for example about 0.5 to 5% by weight of the composition.

The antioxidant can be a sulfite compound, such as potassium metabisulfite, sodium metabisulfite, sodium bisulfite, sodium sulfite, and a mixture thereof. Where sodium metabisulfite is used in a transdermal pramipexole composition, and the composition can exhibit substantially no color change after two weeks at about 60° C.

The antioxidant can also be included in an amount sufficient to enable the composition to exhibit enhanced permeation of the pharmaceutical agent through the dermal or mucosal surfaces compared to a composition not containing the antioxidant.

In an embodiment, the composition also provides sustained release of the pharmaceutical agent, so that the composition can be administered less frequently than an immediate release formulation, about once a day.

### BRIEF DESCRIPTION OF THE DRAWINGS

The features of the invention will be further described in the following detailed description and accompanying drawings in which:

FIGS. 1A-1B graphically illustrate stability of a dopamine agonist as a function of pH under various conditions; and

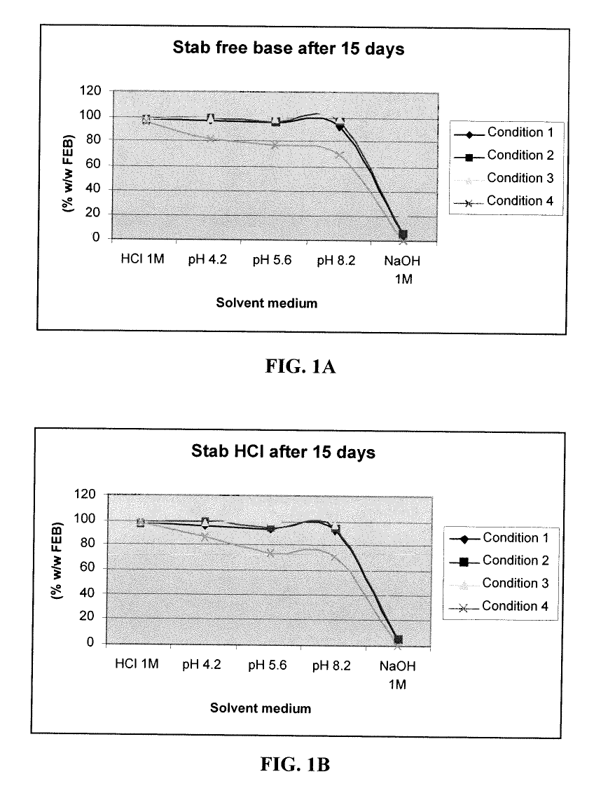

FIGS. 2A-2B compare permeation properties of a pramipexole formulation containing sodium metabisulfite with a pramipexole formulation without sodium metabisulfite.

### DETAILED DESCRIPTION OF PREFERRED EMBODIMENTS

The invention relates to a transdermal composition containing a pharmaceutically active agent effective to treat a neurological disorder, such as pramipexole or a pharmaceutically acceptable salt thereof, which is chemically stable and resistant to color change. The invention also relates to such a pharmaceutical composition, wherein the composition provides continuous and sustained release of the active agent over an extended period of time.

As used herein, the term “anti-Parkinson agent” or “anti-Parkinson drug” is understood to include any drug or pharmaceutically active agent that is effective to treat a neurological disorder such as Parkinson's disease or other such neurological or movement disorders, including Restless Leg Syndrome, Tourette's Syndrome, Chronic Tic Disorder, Essential Tremor, and Attention Deficit Hyperactivity Disorder. Preferably, the anti-Parkinson agent is a dopamine agonist such as pramipexole or other indolone compounds.

As used herein, the term “transdermal” is understood to also include both transdermal and transmucosal delivery of an active agent.

As used herein, the term “color stability” means the ability of a composition to substantially retain its original color during the time of storage or use. The storage time can vary over a period of a few weeks to a month or longer, while the use of the composition can be from one day to one or two weeks. The term “substantially retain” means that there are no visually discernable changes in the color of the formulation over the time of storage or use.

Pramipexole, its chemical structure, processes for its preparation and therapeutic uses thereof are more fully described in U.S. Pat. Nos. 4,452,808, 4,824,860, and 6,770,761, the contents of each of which are expressly incorporated herein by reference. As used herein, the term “pramipexole” includes pharmaceutically acceptable salts thereof. The skilled artisan is well aware of the different types of pharmaceutically acceptable salts that can be selected for formulation and use in the composition. Preferably, pramipexole is used in the form of its hydrochloride or dihydrochloride salt.

As used herein, the amount or concentration of pramipexole is expressed as measured by the free base equivalent of pramipexole.

The composition can be provided in any suitable form for topical application, including a lotion, a cream, a gel, a solution, or a patch, although a non-occlusive form is preferred to eliminate the disadvantages associated with an occlusive form such as patch.

In one embodiment, the invention provides a transdermal composition containing a pharmaceutically active agent effective to treat a neurological disorder, such as pramipexole or a pharmaceutically acceptable salt thereof, which includes an antioxidant to reduce or eliminate chemical and/or color instability problems associated with the active agent.

Active agents such as pramipexole are known to discolor quickly and extensively in a pharmaceutical composition. The discoloration can further depend on the pH of the medium, the composition of the medium, and the nature of the active form, and can range from light to deep discoloration. For example, indolone compounds such as pramipexole are generally more stable at an acidic and neutral pH than at a basic pH, e.g., pH of about 13. While both free base and salt forms of pramipexole appear to have a tendency to undergo hydrolysis, degradation of the free base seems to be further influenced by the reagent. Also, the salt form, e.g., hydrochloride form, is believed to be generally less sensitive to oxidation than the free base form in the presence of an oxidant.

The transdermal composition according to the invention addresses these color and chemical instability problems by including an antioxidant. Advantageously, it has been found that addition of an antioxidant prevents or delays color change and chemical degradation of certain anti-Parkinson agents. Sulfite antioxidants, e.g., sodium metabisulfite (Na₂MET), sodium bisulfite, sodium sulfite, and potassium metabisulfite, are particularly advantageous when used in combination with indolone compounds, with sodium metabisulfite being especially preferred. By including such an antioxidant, the composition provides significantly enhanced short-term and long-term chemical and color stability. For example, in a transdermal composition of pramipexole hydrochloride, the addition of sodium metabisulfite has been shown to completely prevent discoloration and precipitation after two weeks at 60° C., in contrast to a composition not containing such an antioxidant, which exhibited an extensive color change.

This enhanced color stability is useful in that multiple doses of the composition can be packaged together, with the end user applying the composition in desired portions over time. The compositions that contain the antioxidant of the invention do not discolor during a storage time of at least about one week to one month. Thus, for compositions that provide sustained release of the drug for a period of at least one day, a two-week or one-month supply of the dosage forms can be packaged together, purchased and stored without concern of color change that might be interpreted as deterioration of the composition.

The antioxidant can be used in the composition in combination with one or more other antioxidants or chelating agents. Such combination of an antioxidant with another antioxidant or a chelating agent can synergistically increase the benefits of the antioxidant. For example, Na₂MET can be used in combination with, for example, edetic sodium salts, butylhydroxytoluene, butylhydroxyanisole, propyl gallate, ascorbyl palmitate, ascorbic acid, and/or tocopherol.

Advantageously, the antioxidant according to the invention does not adversely affect the permeation properties of the anti-Parkinson agent, but can enhance its permeation properties in addition to providing chemical and color stability. For example, when used in a transdermal pramipexole composition, sodium metabisulfite provides about 30% increase in the skin permeation of pramipexole, as measured by the amount of cumulative drug permeation. Thus, the antioxidant can also function as a permeation enhancer according to the invention.

The composition can additionally contain conventional pharmaceutically acceptable excipients as desired, e.g., non-volatile solvents and thickening agents. The composition can be provided in any dosage form suitable for topical application, including a lotion, a cream, a gel, a solution, or a patch, although a non-occlusive form is preferred to eliminate the disadvantages associated with an occlusive form such as patch.

In one embodiment, the composition provides continuous, sustained release of the pharmaceutically active agent over an extended period of time, to provide a more lasting delivery of the agent than an immediate release formulation. Because therapeutic delivery of the active agent is more sustained, the composition can be administered less frequently than an immediate release formulation. For example, if the composition provides controlled release of the active agent over 24 hours, the composition can be administered once a day without compromising its therapeutic benefits. The composition can be formulated to provide sustained release for a shorter or longer duration by adjusting the amounts of the active agent and the excipients.

The composition can be administered in any desired amount and frequency. The amount and frequency of dosage will depend on the type and amount of the active agent to be administered and the patient's needs, and can be easily adjusted based on the desired total amount of application, severity of the disease, and efficacy of the drug. For example, where the active agent is pramipexole, pramipexole can be present in an amount up to about 60 mg, and preferably about 15 mg to 30 mg, measured as the amount of pramipexole free base equivalent. The concentration of pramipexole in the composition can be, for example, about 0.5 and 5% by weight.

The composition of the invention can be administered in any suitable amount and manner as desired. For instance, the composition can be administered in a single or multiple applications per day, to any desired dermal or mucosal surface area, e.g., about 50 to 1000 cm². The end user will appreciate ease and flexibility of application, as the composition can be applied in any desired dosage on any suitable dermal or mucosal surface.

### EXAMPLES

The invention is further illustrated in the following examples, which are provided for the purpose of illustration only and do not limit the invention in any way.

### Example 1

### Effect of pH on degradation of dopamine agonist

Degradation of a commonly-used dopamine agonist, in free base and hydrochloride forms, at various pH was evaluated under the following four conditions. The results are shown in Tables 1 and 2.

Condition 1: ambient temperature, at dark

Condition 2: ambient temperature, exposed at day light

Condition 3: 5° C. (at dark)

Condition 4: 60° C. (at dark)

TABLE 1
Stability of a dopamine agonist in free base form as a function of pH

| | | HCl 1M | pH 4.2 | pH 5.6 | pH 8.2 | NaOH 1M |
|---|---|---|---|---|---|---|
| T0 | | 96.74 | 98.52 | 98.29 | 97.78 | 92.99 |
| After 15 | Condition 1 | 97.65 | 96.51 | 95.50 | 92.46 | 4.10 |
| days | Condition 2 | 97.48 | 98.45 | 96.31 | 96.54 | 4.90 |
| | Condition 3 | 98.13 | 98.57 | 98.83 | 97.97 | 22.48 |
| | Condition 4 | 96.43 | 82.15 | 77.44 | 69.07 | 0.00 |

TABLE 2
Stability of a dopamine agonist in hydrochloride form as a function of pH

| | | HCl 1M | pH 4.2 | pH 5.6 | pH 8.2 | NaOH 1M |
|---|---|---|---|---|---|---|
| T0 | | 97.65 | 98.46 | 98.51 | 97.69 | 93.58 |
| After 15 | Condition 1 | 97.84 | 95.63 | 93.46 | 92.41 | 4.58 |
| days | Condition 2 | 98.27 | 99.49 | 94.49 | 94.73 | 4.94 |
| | Condition 3 | 97.87 | 97.81 | 99.55 | 97.25 | 27.18 |
| | Condition 4 | 97.58 | 86.54 | 73.76 | 71.72 | 0.00 |

The dopamine agonist, in both free base and hydrochloride forms, was stable under acidic pH but prone to degradation under increased pH in 1M NaOH (pH≈13). However, at pH up to about 8, stability remained satisfactory with only about 8% degradation, as opposed to full degradation in NaOH after 15 days at ambient temperature.

In addition, it was shown that the HCl form is less sensitive to oxidation than the free base form in the presence of an oxidant, with about 94% HCl recovered versus 76.1% free base after 15 days at ambient temperature. The degradation of the free base also seemed to be reagent limited, based on the steady state observed for the free base. Generally, good stability was observed in the dilution solvent and in the receptor solution selected for chromatographic analysis and for permeation study. Further, in the absence of oxygen (e.g., when bubbled with nitrogen), moderate degradation was observed for both the HCl and the base forms, thus indicating a tendency to undergo hydrolysis.

### Example 2

### Screening of Antioxidants

The LC-MS structure elucidation analysis has demonstrated that coloration of an anti-Parkinson dopamine agonist, e.g., pramipexole, is linked to the degradation of the drug. Thus, coloration can be used as surrogate to assess stability of formulations containing such anti-Parkinson agents. A preliminary study was performed to evaluate the effects of various antioxidants and chelating agents on coloration of formulations containing a dopamine agonist.

The following materials were screened:

Edetic acid (EDTA)

Butylhydroxytoluene (BHT)

Propyl gallate (ProGL)

Sodium metabisulfite (Na₂MET)

Edetic acid and edetates are chelating agents commonly considered as antioxidant synergists. BHT, ProGL and Na₂MET are considered as true antioxidants.

The testing levels were selected based on the concentrations normally used in pharmaceutical applications (see HANDBOOK OF PHARMACEUTICAL EXCIPIENTS, 4th ed. (Pharmaceutical Press/American Pharmaceutical Association, 2003)) and compatibility or solubility of the drug with ATD™ media.

An experimental stability plan was designed based on the following scheme:

| | Stabilizing system | | | |
|---|---|---|---|---|
| | EDTA | BHT | ProGL | Na₂MET |
| Sample | 0.10% wt | 0.10% wt | 0.10% wt | 0.10% wt |
| Solution 1 | X | | | |
| Solution 2 | | X | | |
| Solution 3 | | | X | |
| Solution 4 | | | | X |
| Solution 5 | X | X | | |
| Solution 6 | X | | X | |
| Solution 7 | X | | | X |
| Solution 8 | | X | X | |
| Solution 9 | | X | | X |
| Solution 10 | | | X | X |

Sample solutions containing the active ingredient (3.00% dopamine agonist in hydrochloride form) and the antioxidants and/or chelating agents were stored at 60° C. for 10 days in sealed transparent glass-vials. This high storage temperature was selected to facilitate differentiation of the formulations. After 10 days under the specified storage conditions, the sample solutions were visually inspected for color and solubility, which is indicated by precipitation or transparency. The coloration of the solutions was out of the range according to the European Pharmacopoeia. The results are summarized in Table 3 below.

TABLE 3
Coloration and Transparency of Sample Solutions

| Sample | Color | Solubility |
|---|---|---|
| Reference | Dark red | Transparent |
| Solution 1 | Orange | Transparent |
| Solution 2 | Orange/Light red | Transparent |
| Solution 3 | Dark red | Transparent |
| Solution 4 | Dark yellow/Orange | Transparent |
| Solution 5 | Orange | Transparent |
| Solution 6 | Orange | Transparent |
| Solution 7 | Light yellow | Opalescent (*) |
| Solution 8 | Dark red | Transparent |
| Solution 9 | Dark yellow/Orange | Transparent |
| Solution 10 | Yellow | Transparent |

(*) A white precipitate was formed, but was easily re-dispersed by shaking.

The results showed that EDTA, BHT, and ProGL, whether used by itself or in combination, have a significant positive effect on preventing the coloration of the tested active ingredient over time. Na₂MET, in contrast, is shown to prevent or delay the coloration. The preventive effect of Na₂MET becomes more obvious when Na₂MET is used in combination with another antioxidant or chelating agent. A slight synergistic effect was observed when Na₂MET was combined with ProGL, and the least coloration was obtained when Na₂MET was combined with EDTA. However, the combination of Na₂MET and EDTA may not be desirable because of their solubility incompatibility.

### Example 3

### The effect of sodium metabisulfite on pramipexole stability

It is well known that pramipexole dihydrochloride salt is unstable in commonly used pharmaceutical solvents and exhibits rapid, massive discoloration ranging from light to dark yellow. Pramipexole dihydrochloride salt becomes yellowish in semi-solid and liquid formulations upon accelerated aging. The following experiment was performed to assess the effects of antioxidants and chelating agents on coloration of pramipexole hydrochloride formulations.

Various formulations containing 2.00% wt pramipexole hydrochloride (expressed as free base equivalent) were prepared, each containing one of the following agents: edetic sodium salts, butylhydroxytoluene, butylhydroxyanisole, propyl gallate, ascorbyl palmitate, ascorbic acid, tocopherol, and sulfites. The concentration of each agent was as recommended in the HANDBOOK OF PHARMACEUTICAL EXCIPIENTS (4th ed.). A formulation free of antioxidants served as a blank reference. The formulations were placed in crimpled glass vials in a mini-oven (Shel Lab vacuum oven, Sheldon Manufacturing, Inc., Cornelius, Oreg., U.S.A.), the temperature of which was set at 60° C., for 2 weeks, and were then visually examined for color.

The blank formulation turned strongly colored. None of the formulations containing an antioxidant presented a visually significant improvement with respect to color or solubility when compared to the blank reference, except for the formulations containing BHT or a sulfite. Two formulations (Formulations A and B), one containing butylhydroxytoluene and the other containing sodium metabisulfite, were therefore further tested. The formulations were prepared as shown below.

| | FORMULATION | |
|---|---|---|
| | Formulation A | Formulation B |
| Composition | % w/w | % w/w |
| Pramipexole dihydrochloride (as FBE) | 2.00 | 2.00 |
| Permeation enhancing system | 21.00 | 21.00 |
| Ethanol, absolute | 40.00 | 40.00 |
| pH adjusting agent (qs pH 8.0) | 11.50 | 11.50 |
| Butylhydroxytoluene | 0.10 | — |
| Sodium metabisulfite | — | 0.40 |
| Purified water | qs 100.00 | qs 100.00 |

After 2 weeks at about 60° C. in crimped glass vials free of headspace, the formulations were visually inspected. Both were acceptable in term of visual aspects, with no crystallization or precipitation visible. The formulations were also tested for color, following the method of European Pharmacopoeia (5th ed.) (Chapter 2.2.2, § Method I). The formulation with butylhydroxytoluene was ranked as Y2 colored, while, remarkably, the formulation with sodium metabisulfite remained completely colorless. Sodium metabisulfite therefore appears as the preferred stabilisant for pramipexole.

### Example 4

### The Effect of Lowest Effective Concentration of Sodium Metabisulfite on Pramipexole Stability

Various formulations of 2.00% wt pramipexole hydrochloride (expressed as free base equivalent) were prepared, each containing sodium metabisulfite at increasing concentrations: 0.1% wt (Formulation C); 0.2% wt (Formulation D); 0.3% wt (Formulation E) and 0.4% (Formulation B). A formulation free of antioxidants served as a blank reference (Formulation F). The formulations were placed in crimpled glass vials in a mini-oven (Shel Lab vacuum oven, Sheldon Manufacturing, Inc., Cornelius, Oreg., U.S.A.), the temperature of which was set at 60° C., for 2 weeks, and were then visually examined for color.

| | FORMULATION | | | | |
|---|---|---|---|---|---|
| | Formulation F | Formulation C | Formulation D | Formulation E | Formulation B |
| Composition | % w/w | % w/w | % w/w | % w/w | % w/w |
| Pramipexole dihydrochloride (as FBE) | 2.00 | 2.00 | 2.00 | 2.00 | 2.00 |
| Permeation enhancing system | 26.00 | 26.00 | 26.00 | 26.00 | 26.00 |
| Ethanol, absolute | 40.00 | 40.00 | 40.00 | 40.00 | 40.00 |
| pH adjusting agent (qs pH 8.0) | 8.00 | 8.00 | 8.00 | 8.00 | 8.00 |
| Sodium metabisulfite | — | 0.10 | 0.20 | 0.30 | 0.40 |
| Hydroxypropylcellullose | 1.50 | 1.50 | 1.50 | 1.50 | 1.50 |
| Purified water | qs 100.00 | qs 100.00 | qs 100.00 | qs 100.00 | qs 100.00 |

The blank formulation (F) turned strongly colored (“more colored than Y1”). The formulation containing the lowest level of sodium metabisulfite (0.10% wt, formulation C) was ranked “Y2”. All the other formulations were colorless.

After 1 month storage at 60° C., coloration of formulation C worsened to “Y1”. All the other formulations remained colorless.

In parallel, samples stored at 40° C. in a climatic chamber (C-20/600, Climat Temperatur System, Hechingen, Germany) for 1 month were assessed for color as well. The blank formulation (F) was “more colored than Y1”. The formulation containing the lowest level of sodium metabisulfite (0.10% wt, formulation C) was ranked “BY2”. All the other formulations were colorless.

This experiment proves that 0.2% wt is the minimal amount of sodium metabisulfite preventing color degradation of a pramipexole hydrochloride 2.00% (expressed as free base equivalent) hydro-alcoholic mixture at apparent pH around 8.0.

### Example 5

### The Effect of pH on Pramipexole Stability

One formulation of 2.00% wt pramipexole hydrochloride (expressed as free base equivalent) containing sodium metabisulfite 0.2% wt adjusted to about pH 8.0 was compared to a formulation of 2.00% wt pramipexole hydrochloride (expressed as free base equivalent) containing sodium metabisulfite 0.2% wt adjusted to about pH 5.0. The formulations were placed in crimpled glass vials in a climatic chamber at 40° C. (C-20/600, Climat Temperatur System, Hechingen, Germany) and in a mini-oven (Shel Lab vacuum oven, Sheldon Manufacturing, Inc., Cornelius, Oreg., U.S.A.) at 60° C. Formulations were then visually examined for color after one-month storage. All samples were still colorless.

Samples were then further analyzed by HPLC for assay of pramipexole and for pramipexole related substances and degradation products. Pramipexole was first extracted in a mixture of phosphate buffer and methanol and separated on a C18 reversed phase column (Phenomenex Gemini RP 18, 4.6×150 mm, 5 μm) using as mobile phase a mixture of 1.75 g/L K2HPO4 solution adjusted to pH 10.5 with NaOH 1M and methanol (80:20) and running in gradient mode with increasing methanol content up to 85%. Flow rate was 1.0 mL/min. Temperature was 30° C. Injection volume was 25 μL. Detection was carried out by UV at 262 nm.

After one-month storage at 40° C., the pramipexole formulation with pH 5 exhibited a loss of about 4.6% pramipexole (from 98.1% to 93.5% of theory). The impurity profile comprised 11 peaks (note: only impurities with an area percent relative to total area superior to 0.1% were reported) accounting for about 7.1% (area percent relative to total area) relative to pramipexole. The pramipexole formulation with pH 8 exhibited a loss of only about 2.2% pramipexole (from 97.2% to 95.0% of theory). The impurity profile comprised 9 peaks (note: only impurities with an area percent relative to total area superior to 0.1% were reported) accounting for only about 3.4% (area percent relative to total area) relative to pramipexole.

The same pattern was observed and even emphasized at the 60° C. accelerated-ageing condition. After one-month storage at 60° C., the pramipexole formulation with pH 5 exhibited a loss of about 14.3% pramipexole (from 98.1% to 83.8% of theory). The impurity profile comprised 16 peaks (note: only impurities with an area percent relative to total area superior to 0.1% were reported) accounting for about 17% (area percent relative to total area) relative to pramipexole. The pramipexole formulation with pH 8 exhibited a loss of only about 5.1% pramipexole (from 97.2% to 92.1% of theory). The impurity profile comprised 14 peaks (note: only impurities with an area percent relative to total area superior to 0.1% were reported) accounting for only about 5% (area percent relative to total area) relative to pramipexole.

In light of the aforementioned, it is demonstrated that stabilizing effect of sodium metabisulfite is optimal at neutral to lightly basic pH, e.g. from pH 7.0 to pH 9.0.

### Example 6

### The Effect of Sodium Metabisulfite on Pramipexole Skin Permeation

An in vitro study was performed to assess the effects of sodium metabisulfite on skin permeation properties of a transdermal gel formulation containing pramipexole hydrochloride 1.50% FBE. Formulations were prepared as follows, with Formulation D containing sodium metabisulfite.

| | FORMULATION | |
|---|---|---|
| | Formulation D | Formulation D |
| Composition | % w/w | % w/w |
| Pramipexole dihydrochloride (as FBE) | 2.00 | 2.00 |
| Permeation enhancing system | 26.00 | 26.00 |
| Ethanol, absolute | 40.00 | 40.00 |
| pH adjusting agent (qs pH 8.0) | 8.00 | 8.00 |
| Sodium metabisulfite | — | 0.40 |
| Hydroxypropylcellulose | 1.50 | 1.50 |
| Purified water | qs 100.00 | qs 100.00 |

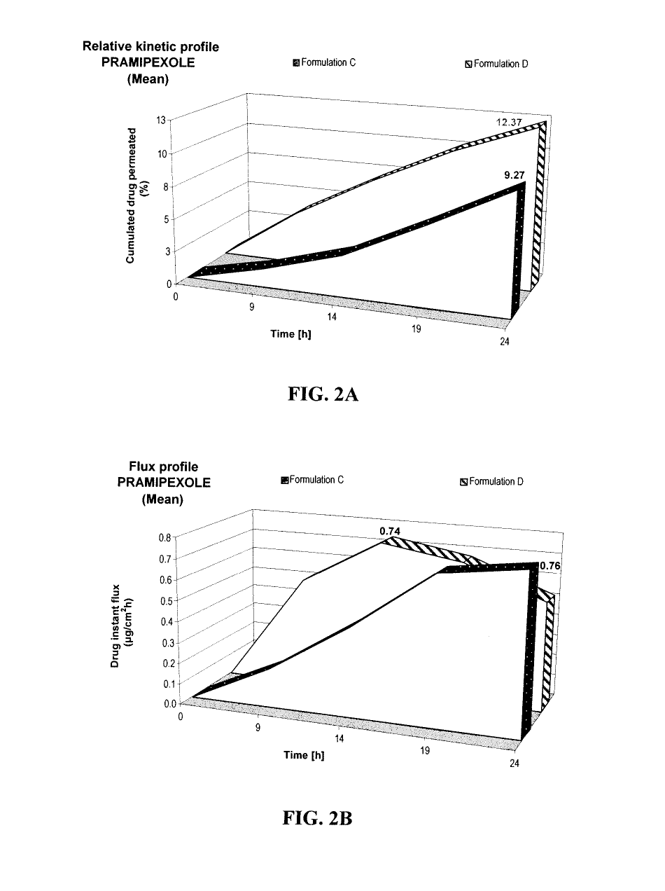

The results are shown in FIGS. 2A and 2B. The data shows that addition of the antioxidant sodium metabisulfite does not impair transdermal bioavailability of pramipexole but actually improves it by about 33%.

What is claimed is:

1. In a transdermal composition that includes a therapeutically effective amount of a pharmaceutical agent effective to treat a neurological disorder, the improvement which comprises including an antioxidant in the composition in an amount sufficient to enable the composition to exhibit enhanced color stability compared to a composition not containing the antioxidant.

2. The composition of claim 1, wherein the composition is non-occlusive.

3. The composition of claim 1, wherein the antioxidant is a sulfite compound.

4. The composition of claim 3, wherein the sulfite compound is potassium metabisulfite, sodium metabisulfite, sodium bisulfite, sodium sulfite, or a mixture thereof.

5. The composition of claim 4, wherein the sulfite compound is sodium metabisulfite and the composition exhibits substantially no color change after not less than one month under extreme accelerated-ageing storage temperature conditions.

6. The composition of claim 4, wherein the temperature is not lower than 40° C.

7. The composition of claim 4, wherein the temperature is not lower than 55° C.

8. The composition of claim 4, wherein the temperature is predictive from long-term stability of the color under ambient temperature condition.

9. The composition of claim 1, wherein the pharmaceutical agent is an indolone compound.

10. The composition of claim 9, wherein the indolone compound is pramipexole, a pharmaceutically acceptable salt of thereof, a pharmaceutically acceptable derivative of thereof, or a mixture thereof.

11. The composition of claim 10, wherein the pramipexole expressed as free base equivalent is present in an amount of about 0.5 to 5% by weight of the composition.

12. The composition of claim 1, wherein the composition provides sustained release of the pharmaceutical agent.

13. The composition according to claim 1, wherein the neurological disorder is Parkinson's disease, Restless Leg Syndrome, Tourette's Syndrome, Chronic Tic Disorder, Essential Tremor, or Attention Deficit Hyperactivity Disorder.

14. In a transdermal composition that includes a therapeutically effective amount of a pharmaceutical agent effective to treat a neurological disorder, the improvement which comprises including an antioxidant in the composition in an amount sufficient to enable the composition to exhibit enhanced permeation of dermal or mucosal surfaces compared to a composition not containing the antioxidant.

15. In a method for treating a neurological disorder by administering to a patient in need thereof a transdermal composition that includes a pharmaceutical agent effective to treat the neurological disorder, the improvement which comprises including an antioxidant in the composition in an amount effective to enable the composition to exhibit enhanced color stability relative to a composition not containing the antioxidant.

16. The method according to claim 15, wherein the composition is non-occlusive.

17. The method according to claim 15, wherein the antioxidant is a sulfite compound.

18. The method according to claim 17, wherein the sulfite compound is potassium metabisulfite, sodium metabisulfite, sodium bisulfite, sodium sulfite, or a mixture thereof.

19. The method according to claim 15, wherein the pharmaceutical agent is an indolone compound.

20. The method according to claim 19, wherein the indolone compound is pramipexole, a pharmaceutically acceptable salt of thereof, a pharmaceutically acceptable derivative of thereof, or a mixture thereof.

21. The method according to claim 20, wherein the pramipexole expressed as free base equivalent is present in an amount of about 0.5 to 5% by weight of the composition.

22. The method according to claim 15, wherein the antioxidant is present in an amount that enables the composition to exhibit enhanced permeation through dermal or mucosal surfaces compared to a composition not containing that amount of antioxidant.

23. The method according to claim 15, wherein the composition is formulated to provide a sustained release of the pharmaceutical agent over at least about 24 hours.

24. The method according to claim 15, wherein the composition is administered once a day.

25. The method according to claim 15, wherein the neurological disorder is Parkinson's disease, Restless Leg Syndrome, Tourette's Syndrome, Chronic Tic Disorder, Essential Tremor, or Attention Deficit Hyperactivity Disorder.
